(19) United States
(12) Patent Application Publication
(10) Pub. No.: US 2024/0238226 A1
(43) Pub. Date: Jul. 18, 2024

(54) IMPLANTABLE SUSTAINED RELEASE DEVICE AND A METHOD OF USE THEREOF

(71) Applicant: Adnan I. Qureshi, Columbia, MO (US)

(72) Inventor: Adnan I. Qureshi, Columbia, MO (US)

(21) Appl. No.: 18/582,169

(22) Filed: Feb. 20, 2024

Related U.S. Application Data
(63) Continuation-in-part of application No. 17/196,953, filed on Mar. 9, 2021, now Pat. No. 11,931,485.

(51) Int. Cl. A61K 31/167; A61K 9/00; A61K 9/16; A61M 5/14

(52) U.S. Cl. CPC A61K 31/167; A61K 9/0092; A61K 9/1647; A61M 5/1413; A61M 2205/0238

(57) ABSTRACT

A biocompatible and bioresorbable implantable device that is intended to be deployed in the arteries supplying the tumors through intra-arterial catheters and release a chemotherapeutic agent for a prolonged period into the branches of the artery to treat tumors. The implantable device may be a mesh with multiple helical loops, fenestrated collapsible hollow shell or spheroid, or folded sheath.

### CROSS-REFERENCE TO RELATED APPLICATIONS

This application is a continuation-in-part of a U.S. patent application Ser. No. 17/196,953 filed on Mar. 9, 2021, which claims priority from a U.S. Provisional Patent Application Ser. No. 63/136,811 filed on Jan. 13, 2021, both of which are incorporated herein by reference in its entirety.

### FIELD OF INVENTION

The present invention relates to a sustained release device and a method of use thereof, and more particularly, the present invention relates to an implantable sustained release device implantable in arteries.

### BACKGROUND

Targeted drug delivery has been a hot research area in the past few decades. Target drug delivery helps to achieve higher drug concentration at site of action while the drug concentration in other areas of the body can be kept low. In cancer treatment, targeted drug delivery has transformed the care of cancer patients. One such method is intra-arterial deliver chemotherapeutics directly to tumors using endovascular approaches. The intraarterial drug delivery approach overcomes the conventional intravenous administration shortcomings, including systemic toxicity, first pass metabolism, and off-target delivery. The intraarterial drug delivery allows for increased local tissue drug levels and a reduction in systemic drug availability. The increase in local drug concentration depends mainly on the blood flow rate of the infusing artery and the rate of drug elimination by the rest of the body. A low arterial blood flow rate and a high drug elimination rate ensures a high local drug level which is often critical. However, it is not just the concentration that results in pharmacodynamic success but also the duration of exposure (i.e., time above bioactive threshold concentration).

New methods for targeted drug deliveries are always desired. Improvements are needed in intraarterial drug deliveries for targeted drug delivery to the tumors.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a sustained release device implantable in arteries for the treatment of tumors.

It is still another object of the present invention that the implantable device can release an active pharmaceutical substance at a sustained rate over a prolonged period.

It is yet another object of the present invention that the implantable device is biocompatible and bioresorbable.

In one aspect, disclosed is a biocompatible and bioresorbable implantable device that is intended to be deployed in the arteries supplying the tumors through intra-arterial catheters and release a chemotherapeutic agent for a prolonged period into the branches of the artery to treat tumors.

In one aspect, the implantable device may be a mesh with multiple helical loops, fenestrated collapsible hollow shell, spheroid, or a folded sheath.

In one aspect, disclosed is an implantable sustain release device that is intended to be deployed in the blood vessel supplying the dura for the treatment of tumor in the dura of the brain.

In one aspect, disclosed is an implantable sustain release device that is intended to be deployed in the blood vessel for the treatment of tumors in the liver or pancreas.

These and other objects and advantages of the embodiments herein and the summary will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 shows a helical implantable sustained release device, according to an exemplary embodiment of the present invention.

FIG. 2 shows the helical implantable sustained release of FIG. 1 in collapsed state within a catheter for implantation, according to an exemplary embodiment of the present invention.

FIG. 3A shows another embodiment of the implantable sustained release device in collapsed state within a catheter for implantation, according to the present invention.

FIG. 3B shows the implantable sustained release device of FIG. 3A partially pushed out of the catheter for illustration, according to an exemplary embodiment of the present invention.

FIG. 3C shows the implantable sustained release device of FIG. 3A in the expanded state, according to an exemplary embodiment of the present invention.

FIG. 4A shows a section of the disclosed implantable sustained release device having the frame, base layer, and a continuous layer of coating, according to an exemplary embodiment of the present invention.

FIG. 4B shows a section of the disclosed implantable sustained release device having the frame, base layer, and a non-corrugate layer of coating, according to an exemplary embodiment of the present invention.

FIG. 5 shows another exemplary embodiment of the implantable sustained release device, according to the present invention.

FIG. 6 shows another exemplary embodiment of the implantable sustained release device, according to the present invention.

### DETAILED DESCRIPTION

Subject matter will now be described more fully hereinafter with reference to the accompanying drawings, which form a part hereof, and which show, by way of illustration, specific exemplary embodiments. Subject matter may however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Disclosed is an implantable sustained release device that is intended to be deployed in the blood vessels that supply the dura mater in the brain. Disclosed is a biocompatible and bioresorbable implantable device that is intended to be deployed in the arteries supplying the tumors through intra-arterial catheters and release a chemotherapeutic agent for a prolonged period into the branches of the artery to treat tumors. It is to be understood that the embodiments have been described for target drug delivery to the dura matter, however, the device and method can be used for any arteries supplying tumors in any part of the body without departing from the scope of the present invention.

The implantable device may be a mesh with multiple helical loops, fenestrated collapsible hollow shell or spheroid, or folded sheath, as shown in the drawings.

Disclosed is a method of targeted drug delivery to a tumor. Also, disclosed is a method for the treatment of tumor. The disclosed device and method can be used for delivering drug to the dura of the brain for treating tumors. Disclosed is a sustained release device that is configured to be implanted in a blood vessel supplying the dura (or other areas of the body). The sustained release device is configured for releasing chemotherapeutic agent in the blood vessel for a prolonged period.

In certain implementations, the sustained release device includes a frame, wherein the frame has an expanded configuration and a collapsed configuration. The frame can transform from the collapsed configuration to the expanded configuration. The frame further includes strands that extend across the lumen within a loop.

In certain implementations, the method includes implanting the sustained release device in the blood vessel by receiving the sustained release device in a collapsed form in arterial catheter. The arterial catheter can be used to position the device in the blood vessel. The device in the collapsed state can be released by pushing out of the arterial catheter with a microwire. The collapsed state can then expand to fit into the blood vessel. In certain implementations, the diameter of the implantable device in the expanded state can be about 1.5-2 times larger than the artery in which it is deployed.

In certain implementation, disclosed is a method for targeted delivery of chemotherapeutic agent. The sustained release device can be implanted in the artery through an arterial catheter. The sustained release device in the collapsed form can be loaded on the arterial catheter. Through, the arterial catheter, the sustained release device can be released into the blood vessel, wherein sustained release device in the blood vessel expands against the blood vessel, wherein the sustained release device is configured to expand to the predefined shape that remain in position in the blood vessel without immediate displacement.

In certain implementations, the sustained release device includes a frame and a coating on the frame, the coating can include the chemotherapeutic agent and a bioabsorbable polymer. The frame can be made of a biodegradable metal or a bioresorbable polymeric material. The frame can be made from material, such as magnesium alloy, polylactic acid, polycarbonate polymers, salicylic acid polymers or combination thereof. The frame can have an additional base layer of a biocompatible material uniformly covering the frame, and one or more layers of the coating is applied on the base layer. The biocompatible material can be poly n-butyl methacrylate, PTFE, PVDF-HFP, poly(styrene-bisobutylene-b-styrene), Parylene C, PVP, PEVA, SBS, PC, TiO2, or a combination thereof.

In certain implementations, the chemotherapeutic agent can be cisplatin, carboplatin, fluorouracil, methotrexate, oxaliplatin, gemcitabine, nafamostat mesiltate, mitomycin, floxuridine, irinotecan, or anti vascular endothelial growth factor agents.

In certain implementations, the sustained release device upon implantation in an artery can release the chemotherapeutic agent for a prolonged period ranging from 1 to 6 months.

In certain implementations, the frame can further have a mesh that extends between the helical wire loops, wherein the mesh and the helical wire loops assume parallel configuration perpendicular to the arterial wall, wherein the helical wire loops elongate in opposite direction resulting in decrease in a diameter of the frame, which can be inserted in the arterial catheter.

In certain embodiments, the sustained release device may further have a mesh that extends from the frame, the mesh having one or more layers of coating.

The frame can be fenestrated collapsible hollow shell or spheroid, as shown in the drawings.

In certain embodiments, the frame can be a rolled flat sheath, as shown in the drawings, having one or more layers of coating, wherein the rolled flat sheath partly unfolds to expand in the blood vessel.

In certain implementations, disclosed is a method of treatment of recurrent headaches and/or trigeminal neuralgia. The method includes preparing a sustained release device that can be implanted in a blood vessel supplying the dura (or other areas of the body) for targeted drug delivery into the dura matter (or other medical site of the action in the body). The sustained release device can release an anesthetic agent in the blood vessel for a prolonged period. The sustained release device can also include microspheres (100-500 micron in diameter) encapsulating the chemotherapeutic agent. Microspheres may be spherical microscopic particles that range in size from 1-1,000 μm. The chemotherapeutic agent can be released for prolonged duration from the microspheres embedded on the sustained release device in the arteries supplying the tumor.

In certain implementations, also disclosed is a method for sustained release of chemotherapeutic agent in arteries through microspheres. The microspheres, which can be spherical microscopic particles that range in size from 1-1,000 μm and encapsulating chemotherapeutic agent. The microspheres can be suspended in an aqueous solution, such as saline, normal saline with contrast solution, and the like. The suspension can be delivered into an artery through a microcatheter, wherein the microspheres can get trapped into the small arteries. The suspension medium can wash away in the blood vessel leaving behind trapped microspheres. The microspheres degrade over time releasing the chemotherapeutic agent.

### EXAMPLE 1

### Preparation of Microspheres

Dissolving 100 mg polyvinyl alcohol in 10 ml deionized water at 90° C. for 3 hours (solution A, 10 ml), subsequently bringing the polyvinyl alcohol solution to room temperature or 38° C. Separately, dissolving 125 mg Poly(lactic-co-glycolic acid) (PLGA) and 25 mg lidocaine in 2 ml of dichloromethane: ethyl alcohol (3:1) solution; i.e., the solution containing 1.5 ml dichloromethane and 0.5 ml ethyl alcohol, subsequently mixed using vortex at room temperature to prepare solution B (2 ml). Thereafter, solution B (2 ml) was added dropwise to solution A (10 ml) at 500 rpm for 4 hours at 38° C. (using heating plate) or 10,000 rpm for 2.5 mins (using homogenizer). The resulting solution was subjected to evaporation under a vacuum in a rotary evaporator for evaporating dichloromethane. The resultant microspheres were transferred to 15 ml centrifuge tubes and centrifuged at 1500 rpm for 2 min. Then, the supernatant can be discarded, and the pellets (of microspheres) are collected. The formed microspheres were washed thrice with 10 ml distilled water using centrifuge. The pellets were collected and subjected to freeze drying. The prepared microspheres were stored at 4° C.

In certain implementations, disclosed is a method for the treatment of recurrent headaches and/or trigeminal neuralgia using a sustained release device. The sustained release device can be implanted in a blood vessel supplying the dura (or other areas of the body). The implanted sustained release device can release the anesthetic agent in the blood vessel for a prolonged period. The sustained release device may include filaments, which may be solid or hollow. The sustained release device can be pushed through the blood vessel using a microcatheter equipped with a microwire. The filaments can be hollow tube-like structure, made of poly(lactic-co-glycolic acid) (PLGA) using the above-mentioned method. The filaments can be biodegradable, 0.5-2.0 mm thick and 10-40 mm long.

The filaments can be rod like structures having a solid or hollow interior. The filaments can be characterized by their ability to bend and twist without mechanical deformation or breaking. The flexibility is conferred by the material that composes the filaments and thickness of the components. The filaments are usually suspended in aqueous solutions, such as normal saline or normal saline with contrast solution. The filament can be advanced into the microcatheter and then pushed forward through injection of aqueous solution or by a microwire. The filament gets trapped in the small arteries and degrades over time releasing the chemotherapeutic agent.

### EXAMPLE 2

### Preparation of Filaments

250 mg of PGLA and 50 mg of lidocaine in 4 ml of dichloromethane: ethyl alcohol mixture (3:1) were dissolved and mixed using vortex at room temperature. The solution is cast in the hollow filament mold, or alternatively the hollow filament is synthesized using the 3D bio-print or microfluidic system. The filaments were subjected to air drying (drying at room temperature) overnight. The filaments were then placed in vacuum oven for 3 hours at 38° C. The hollow filaments were then stored at 4° C. until further use.

In certain implementation, disclosed is a method of causing vasoconstriction of middle meningeal artery and branches thereof using lidocaine as a therapeutic agent. The lidocaine can be prepared as a sustained release formulation that can be implanted in an artery. The sustained release formulation can be a device that can be implanted in an artery using a microcatheter and microwire. Such as device has been explained above and shown in the drawings. The sustained release device can also be in the form of microspheres or filaments that can be injected in the arteries suspended in normal saline. The sustained release of lidocaine can cause vasoconstriction providing relief in migraine.

In certain implementations, disclosed is a method of treatment of headaches associated with intracranial or subarachnoid hemorrhage. The method includes steps of injecting liquid lidocaine solution (1 mg lidocaine in 25 ml of normal saline or distilled water) as a sustained release formulation in middle meningeal artery through blood vessel. The sustained release formulation can be a device that can be implanted in the middle meningeal artery using a microcatheter and microwire. Such as device has been explained above and shown in the drawings. The sustained release device can also be in the form of microspheres or filaments that can be injected in the middle meningeal artery suspended in normal saline.

In certain embodiment, disclosed is an implantable sustained release device that is intended to be deployed in the blood vessels that supply the dura mater in the brain. The disclosed device can be implanted in the middle meningeal artery for releasing an active pharmaceutical ingredient into the branches of the middle meningeal artery. The middle meningeal artery is the predominant source of blood supply to the dura mater. The middle meningeal artery originates from the internal maxillary artery and enters the skull through the foramen spinosum of the sphenoid bone, where it then divides into anterior and posterior branches. The middle meningeal artery divides into frontal, parietooccipital, and posterior temporal branches which are approx. 400-800 μm in diameter. The middle meningeal artery is one of the main arterial supplies, although the accessory middle meningeal artery may also supply the trigeminal nerve ganglion. The artery to trigeminal nerve ganglion has been identified as a branch that arises from the extracranial segment of the middle meningeal artery before entry into the foramen spinosum.

The active pharmaceutical ingredient can be an anesthetic agent for the treatment of brain disorders such as headaches, migraine, facial pain, and trigeminal neuralgia. The device can release the anesthetic agent for a prolonged period, such as 3-6 months to treat severe headaches including migraine and trigeminal neuralgia. The anesthetic agent can be lidocaine, bupivacaine, or the like pharmacological agent. The anesthetic agent can be provided as a coating in the disclosed implantable device. The coating includes carrier polymers which provide for sustained release of the anesthetic agent. The anesthetic agent can be dispersed in a polymeric carrier and the mixture can be applied as layers on a frame of the disclosed implantable device. Multiple layers can be provided on the frame that allows sustained release of the anesthetic agent over a prolonged period. The polymeric carrier can preferably be a bioabsorbable polymer. Bioabsorbable polymers for use in medicines are known to a skilled person, and any such bioabsorbable polymers are within the scope of the present invention.

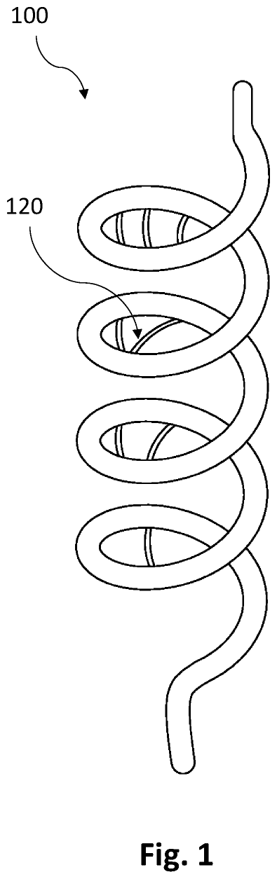

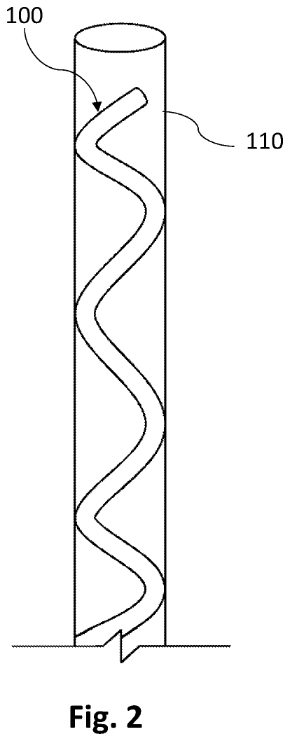

Referring to FIG. 1, which shows an exemplary embodiment of the disclosed implantable device 100 which has a helical profile. The implantable device 100 includes a frame that can be resilient and switch between an expanded state and a collapsed state. FIG. 2 shows the implantable device 100 in the collapsed state while FIG. 1 shows the implantable device 100 in the expanded state. The disclosed implantable device can be deployed in the blood vessel using an arterial catheter 110 and the implantable device can be received in the collapsed form into the catheter.

The implantable device can be implanted into the blood vessel, such as the middle meningeal artery using an arterial catheter 110. In the middle meningeal artery, the disclosed implantable device can be deployed through an arterial route such as a femoral or radial artery. The implantable device, in the collapsed form, can be deployed in the arterial microcatheter, which can then be inserted through the artery up to the desired location i.e., the middle meningeal artery. The device can then be pushed out of the catheter with a standard microwire until the implantable device is extruded from the microcatheter into the blood vessel. The device in the blood vessel can expand and fit into the blood vessel. The implantable device by virtue of its biomechanical properties assumes the predefined shape that ensures that the implantable device remains in position in the artery without immediate displacement. In one exemplary embodiment, the diameter of the implantable device in the expanded state is no more than 2 mm.

In one exemplary embodiment, for deploying the disclosed implantable device, the procedure can be performed after arterial access is achieved by placement of a sheath in the femoral arterial or radial artery. The external carotid artery is catheterized using a 5 F or 6 F catheter. Images are obtained in anteroposterior and lateral planes and the absence of any Dural arteriovenous fistula or anastomoses between external and internal carotid artery branches can be confirmed. After confirmation, a single lumen microcatheter can be advanced over a 0.014 in. microwire through the 5 F or 6 F catheter into the external carotid artery. Under fluoroscopic guidance, the microcatheter can be advanced through the proximal internal maxillary artery and into the middle meningeal artery. A microcatheter injection can be performed in both anteroposterior and lateral planes to visualize the Dural branches of the middle meningeal artery and the absence of any contribution to ophthalmic or intracranial arteries.

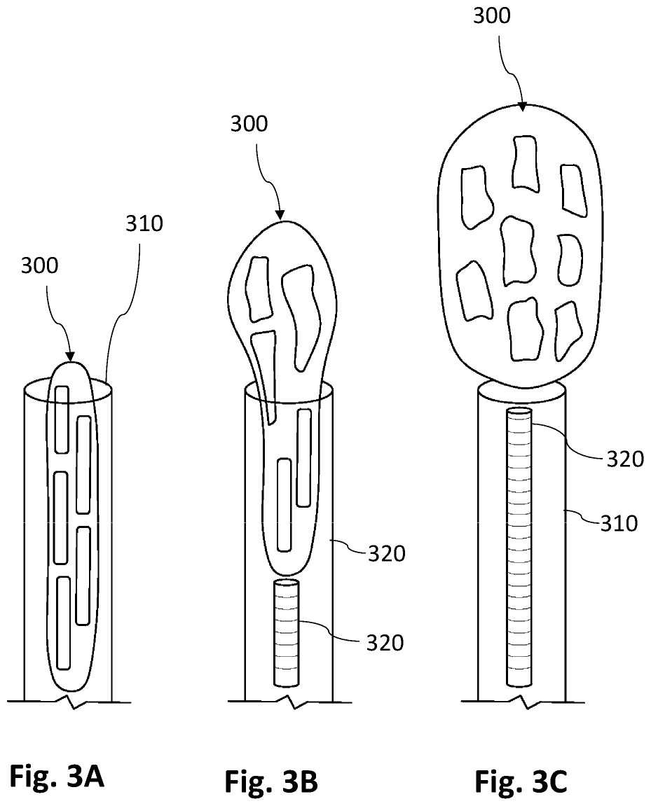

The implantable device can be introduced through the exterior end of the microcatheter and pushed through the lumen using any 0.014 in. microwire. Once the implantable device reaches the distal end of the microcatheter, the device can be deployed by either pushing it forward or retracting the microcatheter with constant forward push of the delivery microwire. FIG. 3A shows another exemplary embodiment of the implantable device which is fenestrated collapsible hollow shell 300. FIG. 3A shows device 300 in collapsed state inserted in the distal end of catheter 310. FIG. 3B shows device 300 being pushed out of the catheter 310 using a microwire 320. The portion of device 300 coming out of the catheter is being expanded. FIG. 3C shows the device 300 pushed out of the catheter 310 and expanded to the expanded state. FIG. 1 shows the helical loop-shaped device 100 in the expanded state. The frame of the device can have biomechanical properties by virtue of which the device can expand against the blood vessel and stay in place in the blood vessel.

The frame acts as a carrier for the active pharmaceutical agent, such as an anesthetic agent, which can be implanted in the blood vessel. The frame can be made from any biodegradable metal or polymer, such as magnesium alloy, polylactic acid, polycarbonate polymers, salicylic acid polymers, and/or combinations thereof. Coating of the pharmaceutically active agent can be applied directly on the frame, or a base layer can be applied to the frame first.

The base layer can be of a biocompatible material that can cover the entire frame. For example, the biocompatible base layer may be made from poly n-butyl methacrylate, PTFE, PVDF-HFP, poly(styrene-bisobutylene-b-styrene), Parylene C, PVP, PEVA, SBS, PC, TiO2, or any material that has good biocompatibility. The coating containing the active pharmaceutical agent can be coated as one or more layers on the biocompatible base layer.

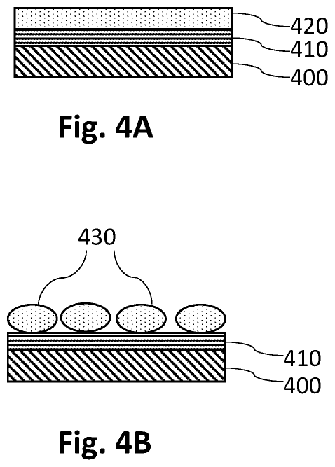

The coating including the active pharmaceutical agent provides for sustained release of the active pharmaceutical agent. The coating can include a bioabsorbable polymer into which the active pharmaceutical agent can be dispersed. The device can include multiple layers of coating for sustained release. The release of the active agent depends on the total surface area of the frame. The surface area can be increased by providing a mesh 120 made from strands interconnecting the helical loops, shown in FIG. 1. The coating can be uniformly applied over the helical loops and the mesh. Alternatively, the coating can also be applied as a layer in non-congruent portions. FIG. 4A is a sectional view of the device showing frame 400 having the first base layer 410 and a coating 420 containing the anesthetic agent. FIG. 4B is the sectional view of the device showing the frame 400 having the uniform first base layer 410 and a coating 430 is applied as a layer of non-congruent portions.

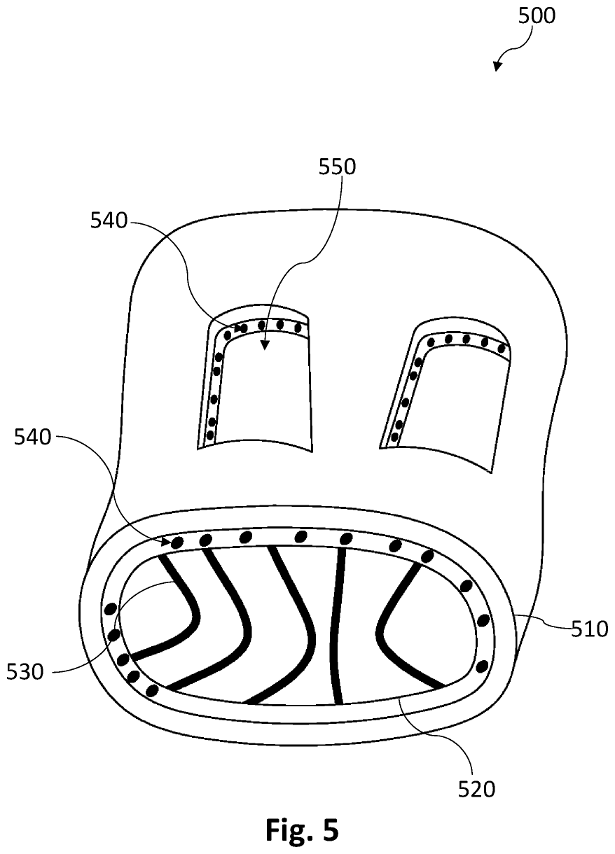

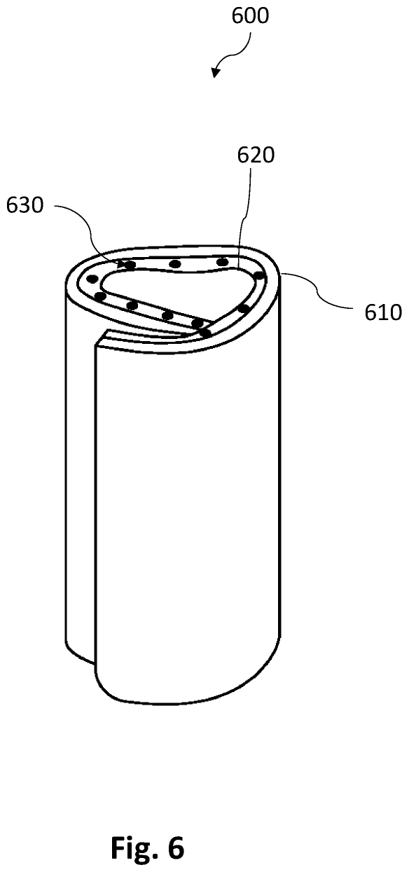

The frame of the disclosed device can embody different geometric forms that can be collapsed under external force and expands to their shape when the external force is removed. FIG. 1 shows the device in the form of helical loops having mesh. FIG. 3C shows the disclosed device as a fenestrated collapsible hollow spheroid. FIG. 5 shows the device 500 in the form of the fenestrated collapsible hollow shell. The device 500 having a shell 510 having an inner coating 520 and mesh 530. Anesthetic agent 540 is dispersed in the coating. The shell having windows 550 for increased surface area. FIG. 6 shows the disclosed device 600 in the form of a folded sheath. The device 600 has the frame 610, a coating 620 over the frame, and an anesthetic agent 630.

### EXAMPLE 3

The effects of intra-arterial injection of a dose of 40 mg lidocaine and 20 mg methylprednisolone into the middle meningeal artery of two patients suffering from severe headaches were determined.

It was observed that the effect of injection of lidocaine and methylprednisolone was short-lasting with effects manifesting within 5 min and lasting 5-8 h after injection. Both patients reported improvement in headache intensity after 24 h post-procedure. Microcatheter injection was performed in both anteroposterior and lateral planes to visualize the Dural branches of the middle meningeal artery and the absence of any contribution to ophthalmic or intracranial arteries. A dose of 40 mg lidocaine (2 mg/ml dilution in normal saline) injected in 10 mg doses was administered over 5 min into the middle meningeal artery. Subsequently, 20 mg methylprednisolone (4 mg/ml dilution in normal saline) was injected over 5 min into the middle meningeal artery. Heart rate and single-lead EKG were continuously monitored, and blood pressure was monitored using an automated cuff every 3 min. The patient reported improvement in headache from a self-reported intensity of 10 (on Visual Analog scale) to 5 after 5 min of lidocaine injection. At a subsequent interview at 2.5 h post-procedure, the patient reported that headache was better than pre-procedure headache with intensity rated at 6.5. The patient reported the recurrence of headache at 8 h with intensity rated at 8. After 24 h, the patient reported an improvement in headache with intensity rated at 5. The patient appeared to be more comfortable and was discharged. On the day of discharge, the patient was resting comfortably but denied any persistent headache. The patient reported improvement in headache from a self-reported intensity of 7 (on Visual Analog scale) to 4 after 5 min of lidocaine injection. The patient reported recurrence of headache after 5 h to a self-reported intensity of 7. The patient reported complete resolution of headache after 12 h and mild headache at 16 h post-procedure.

### EXAMPLE 4

Trigeminal neuralgia consists of unilateral, brief/paroxysmal, or continuous pain in one or more divisions of the trigeminal nerve. The pain is secondary to hyperactivity or spontaneous impulse generation within the nerves due to extrinsic compression or intrinsic neural dysfunction. In the experiment, the intra-arterial delivery of medication for modulating trigeminal nerve ganglion function in patients with refractory trigeminal neuralgia was examined. Was administered intra-arterial lidocaine in doses up to 50 mg in the middle meningeal artery territory adjacent to the arterial branch that supplies the trigeminal nerve ganglion. Electrophysiologic monitoring was performed to serially assess the latency and amplitude of R1 and R2 responses in the blink reflex before and concurrent with each incremental dose of lidocaine. Clinical outcome assessment included a 10-point numeric rating, 4-point severity grading, and the pain-free time interval pre-and post-treatment. Intra-arterial lidocaine was administered to three patients with trigeminal neuralgia (35-year-old woman, 57-year-old man, and a 34-year-old woman). In all patients, there was a latency prolongation and amplitude reduction of R1 or R2 responses or both which was evident after 5-10 mg of lidocaine administration; a more pronounced effect was seen with increasing doses. The second and third patients reported improvement in pain severity on all scales with pain-free intervals of 5 and 3 days, respectively. There was an improvement in facial hyperalgesia in all three patients in all dermatomes. All three patients' symptoms had returned to baseline severity 1 month later. It was found that modulation of trigeminal nerve activity via the intra-arterial route is possible based on consistent intraprocedural electrophysiologic suppression and short-term clinical improvement in patients with refractory trigeminal neuralgia.

By blocking sodium channels, lidocaine stabilizes nerve membranes, delays nerve depolarization, and reduces ectopic discharges. The elimination half-life of lidocaine following an intravenous bolus injection was found to be typically 1.5-2 hours.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method of sustained release of a chemotherapeutic agent in an artery, the method comprising:
preparing a sustained release device configured to be implanted in an artery through an arterial catheter,
loading a chemotherapeutic agent on the sustained release device, wherein the sustained release device is configured to release the chemotherapeutic agent for a prolong period.

2. The method of claim 1, wherein the sustained release device comprises a frame, wherein in the frame is configured to switch from a collapsed configuration to an expanded state upon implantation, wherein the frame is formed as a wire curved in spaced apart loops forming an elongated helical wire profile, wherein the frame further comprises strands that extend across a lumen of the frame and within a loop.

3. The method of claim 2, wherein the method further comprises:
implanting the sustained release device in the artery, wherein the sustained release device in the collapsed form is released from the arterial catheter by a microwire, and wherein the sustained release device expands into the artery, wherein a diameter of the sustained release device in the expanded state is 1.5-2 times larger than a diameter of the artery in which it is deployed.

4. The method of claim 2, wherein the method further comprises:
receiving the sustained release device, in a collapsed form, in the arterial catheter; and
releasing the collapsed sustained release device into the artery, wherein the sustained release device in the artery expands against a wall of the artery, wherein the sustained release device is configured to expand to a predefined shape that remain in position in the artery without immediate displacement.

5. The method according to claim 4, wherein the sustained release device comprises aa coating on the frame, the coating comprises the chemotherapeutic agent and a bioabsorbable polymer.

6. The method according to claim 5, wherein the frame is made of a biodegradable metal or a bioresorbable polymeric material.

7. The method according to claim 1, wherein the chemotherapeutic agent is selected from a group consisting of cisplatin, carboplatin, fluorouracil, methotrexate, oxaliplatin, gemcitabine, nafamostat mesiltate, mitomycin, floxuridine, irinotecan, and anti vascular endothelial growth factor agents, and wherein the sustained release device is implanted for a treatment of tumor.

8. The method of claim 7, wherein the sustained release device is implanted in the artery supplying dura for the treatment of tumors.

9. The method according to claim 1, wherein the prolonged period ranges from 1 to 6 months.

10. The method according to claim 2, wherein the frame further comprises a mesh that extends between the helical wire loops, wherein the mesh and the helical wire loops assume parallel configuration perpendicular to an arterial wall in which the sustained release device deployed, wherein the helical wire loops elongate in opposite direction resulting in decrease in a diameter of the frame, allowing it to be inserted into the arterial catheter.

11. The method according to claim 10, wherein the mesh comprises one or more layers of coating, the coating comprises the chemotherapeutic agent.

12. The method according to claim 1, wherein the sustained release device comprises a frame, wherein in the frame is configured to switch from a collapsed configuration to an expanded state upon implantation, wherein the frame is fenestrated collapsible hollow shell or spheroid.

13. The method according to claim 1, wherein the sustained release device comprises a frame, wherein in the frame is configured to switch from a collapsed state to an expanded state upon implantation, wherein the frame is a rolled flat sheath having one or more layers of a coating, the coating comprises the chemotherapeutic agent, wherein the rolled flat sheath is configured to partly unfolds to expand in a blood vessel.

14. The method of claim 1, wherein the sustained release device is in a form of microspheres, wherein the microspheres are suspended in a saline solution and delivered in the artery through the artery catheter.

15. The method according to claim 14, wherein the chemotherapeutic agent is an anesthetic agent for a treatment of recurrent headaches and/or trigeminal neuralgia, wherein the microspheres are implanted in the arteries supplying dura of brain, wherein the sustained release device is configured to release the anesthetic agent in arteries for a prolonged period.

16. The method of claim 15, wherein the step of preparing the microspheres and loading the chemotherapeutic agent comprises:
dissolving 100 mg polyvinyl alcohol in 10 ml deionized water at 90° C. for 3 hours and subsequently allowing prepared polyvinyl alcohol solution to cool down to room temperature to obtain solution A;
dissolving 125 mg poly(lactic-co-glycolic acid) and 25 mg lidocaine in 2 ml of dichloromethane:ethyl alcohol (3:1) solution to obtain solution B;
adding 2 ml of solution B, dropwise, to 10 ml of solution A with mixing for 4 hours at 38° C. and thereafter 2.5 mins under homogenization; and
removing dichloromethane under vacuum to obtain the microspheres.
